(19) United States
(12) Patent Application Publication
(10) Pub. No.: US 2025/0255689 A1
(43) Pub. Date: Aug. 14, 2025

(54) SURGICAL DRAPE

(71) Applicant: MEDENVISION, Westerlo (BE)

(72) Inventors: Pieter DEBOECK, Tielt-Winge (BE); Filip VAN LIMBERGEN, Hulshout (BE); Wouter FOULON, Lubbeek (BE); Kristoff CORTEN, Wolfsdonk (BE); Floris GOYENS, Antwerpen (BE)

(73) Assignee: MEDENVISION, Westerlo (BE)

(21) Appl. No.: 18/439,586

(22) Filed: Feb. 12, 2024

(51) Int. Cl. A61B 46/27; A61B 46/20

(52) U.S. Cl. CPC A61B 46/27; A61B 2046/201

(57) ABSTRACT

The present invention relates to a surgical drape for shoulder surgery. The surgical drape comprises a main covering layer having a front surface and a back surface, an absorbing layer applied to the front surface wherein a first cut is made through the absorbing layer and the main covering layer, the cut being configured that the drape can move over an arm through the cut, a limb sleeve having a closed end and an open end wherein the limb sleeve is assembled to the main covering layer at the open end and the cut aligned such that when the drape is moved over the arm through the cut, the arm is covered by the limb sleeve, and a tourniquet element having an elastic opening configured to move over an arm with a circumferential pressure on the arm, wherein perforations are provided in the limb sleeve for tearing off part of the limb sleeve and wherein the tourniquet element is assembled in the limb sleeve at a position between the open end of the limb sleeve and the perforations.

### FIELD OF THE INVENTION

The present invention relates to a surgical drape and to methods for using a surgical drape for preparing a patient for surgical procedure.

### BACKGROUND OF THE INVENTION

Surgical drapes are known. They are often not easy to use and require multiple surgical drapes especially for surgical procedures at difficult areas such as for example the shoulder.

Especially shoulder surgery requires today a lot of time to prepare the patient for the surgical procedure. The patient is covered by multiple sterile drapes until only the area needed of the surgical procedure is not covered.

There is a need for a surgical drape which allows easier preparation for a shoulder surgical procedure. Next to a surgical drape, there is a further need for a method to prepare the patient in an easier, more predictable and faster way for surgery on the shoulder.

### SUMMARY OF THE INVENTION

It is an aim of the present invention to provide a surgical drape which overcomes at least some of the above mentioned problems.

This aim is achieved according to the invention with a surgical drape for shoulder surgery, the surgical drape, comprising a main covering layer having a front surface and a back surface, an absorbing layer applied to the front surface wherein a first cut is made through the absorbing layer and the main covering layer, the cut being configured that the drape can move over an arm through the cut, a limb sleeve having a closed end and an open end wherein the limb sleeve is assembled to the main covering layer at the open end and the cut aligned such that when the drape is moved over the arm through the cut, the arm is covered by the limb sleeve, and a tourniquet element comprising an elastic opening configured to move over an arm and a connection edge assembled in the limb sleeve at a first distance from the open end, wherein perforations are provided in the limb sleeve for tearing off part of the limb sleeve at a second distance from the open end, and wherein the first distance is lower than the second distance such that the tourniquet element is connected in the limb sleeve at a position between the open end of the limb sleeve and the perforations.

It is an advantage of the surgical drape according to the invention that it enables preparing a patient with the surgical drape in a fast way with a minimum risk of sterilization errors because the drape can cover the patient in only a few standardized steps and the perforations allow to create an accessible area without touching the accessible area with hands. It is a further advantage of the present invention, that fewer steps, less time, and less manpower is required in order to cover a patient in a sterile manner, without compromising the freedom of the surgeon to tailor the surgical window to the requirements of the shoulder surgery and patient characteristics.

In some embodiments of the invention, the surgical drape further comprises a reinforcement element, wherein the reinforcement element has a second cut, the second cut being configured such that the drape can move over an arm through the second cut and wherein the reinforcement element is applied to the back surface of the main covering layer with the second cut aligned to the first cut such that the drape can move over an arm through the first cut and the second cut.

The reinforcement element reinforces not only the opening, it also protects the connection of the limb sleeve to the main covering layer resulting in a more reliable drape.

In some embodiments of the invention, the perforations are at a distance between 5 cm and 9 cm from the open edge of the limb sleeve, preferably between 6 cm and 8 cm, and most preferred at 7 cm from the open edge of the limb sleeve.

The location of the perforation with respect to the open end has the advantage that there is sufficient space for assembling the tourniquet element between the open edge and the perforations and at the same time have the perforations close enough to the open end to be able to tear off close to the shoulder.

In some embodiments of the invention, the perforations are provided circumferential around the limb sleeve.

This has the advantage that it is easy to tear off part of the limb sleeve.

In some embodiments of the invention, the surgical drape further comprises a means for indicating the location of the perforations, wherein the means for indicating the location of the perforations is provided on the limb sleeve.

This has the advantage that the user knows well where to tear off part of limb sleeve saving time.

In some embodiments of the invention, the means for indicating the location of the perforations is glued on the limb sleeve.

This has the advantage that it is a trusted way of providing something.

In some embodiments of the invention, the limb sleeve comprises a sleeve starting layer which is folded in the length direction and glued on both sides of the folded parts to form the sleeve with a closed end and an open end, wherein the folded parts are glued up to a first distance from the open end, and wherein the perforations are at a second distance from the open end, wherein the second distance is larger than or equal to the first distance.

This has the advantage that part of the limb sleeve can be easier torn off at the perforations and that the part of the limb sleeve that remains in the drape is still glued together at the sides.

In some embodiments of the invention, the limb sleeve is glued to the main covering layer.

This has the advantage that gluing is a reliable way of assembling preventing the limb sleeve from loosening.

In some embodiments of the invention, the tourniquet element is glued in the limb sleeve.

This has the advantage that gluing is a reliable way of assembling preventing the tourniquet element from loosening.

It is a further aim of the present invention to provide a method of sterile covering of a patient for shoulder surgery which overcomes at least some of the above mentioned problems.

This aim is achieved according to the invention with a method of sterile covering of a patient for shoulder surgery using a surgical drape wherein the surgical drape is folded such that the limb sleeve can unfold without unfolding the main covering layer, the method comprising
moving the surgical drape through the first cut over an arm of a patient causing that the arm is covered by the limb sleeve,
unfolding the main covering layer over the patient,
tearing off part of the limb sleeve at the perforations,
moving the torn off part of the limb sleeve over the arm away from the shoulder to create an accessible area of desired size for the surgery,
fixing the position of torn off part of the limb sleeve on the arm when the desired size for the accessible area is reached.

It is an advantage of the method of sterile covering of a patient for shoulder surgery that the patient is sterile covered in a fast way with a minimum risk of sterilization errors because the drape can cover the patient in only a few steps and the perforations allow to create an accessible area without touching the accessible area with hands. It is a further advantage of the present invention, that fewer steps, less time, and less manpower is required in order to cover a patient in a sterile manner.

In some embodiments of the invention, the fixing of the torn off part of the limb sleeve is executed by applying a tape on the torn off part of the limb sleeve.

This has the advantage that this can be executed fast in a reliable way and that it can be executed with minimum risk of sterilization errors.

Particular and preferred aspects of the invention are set out in the accompanying independent and dependent claims. Features from the dependent claims may be combined with features of the independent claims and with features of other dependent claims as appropriate and not merely as explicitly set out in the claims.

These and other aspects of the invention will be apparent from and elucidated with reference to the embodiments described hereinafter.

### BRIEF DESCRIPTION OF THE DRAWINGS

FIG. 1 illustrates layers of a surgical drape according to an embodiment of the invention.

FIG. 2 illustrates a second part of the main covering layer according to an embodiment of the invention.

FIG. 3 illustrates a sleeve starting layer to form a limb sleeve according to an embodiment of the invention.

FIG. 4 illustrates a limb sleeve according to an embodiment of the invention.

FIG. 5 illustrates a tourniquet element positioned in a limb sleeve according to an embodiment of the invention.

FIG. 6 illustrates a starting layer to form a tourniquet element according to an embodiment of the invention.

FIG. 7 illustrates a reinforcement element to apply to a main covering layer at the opening for a limb according to an embodiment of the invention.

FIG. 8 illustrates a reinforcement element positioned on a main covering layer according to an embodiment of the invention.

FIG. 9 illustrates a surgical drape according to an embodiment of the invention.

The drawings are only schematic and are non-limiting. In the drawings, the size of some of the elements may be exaggerated and not drawn on scale for illustrative purposes. Further, relative dimensions may not correspond to actual reductions to practice of the invention.

Any reference signs in the claims shall not be construed as limiting the scope.

In the different drawings, the same reference signs refer to the same or analogous elements.

### DETAILED DESCRIPTION OF THE INVENTION

The present invention will be described with respect to particular embodiments and with reference to certain drawings but the invention is not limited thereto but only by the claims.

The terms “up”, “down”, “high”, “low”, “horizontal”, “vertical”, “top”, “bottom”, “side” and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

### Surgical Drape

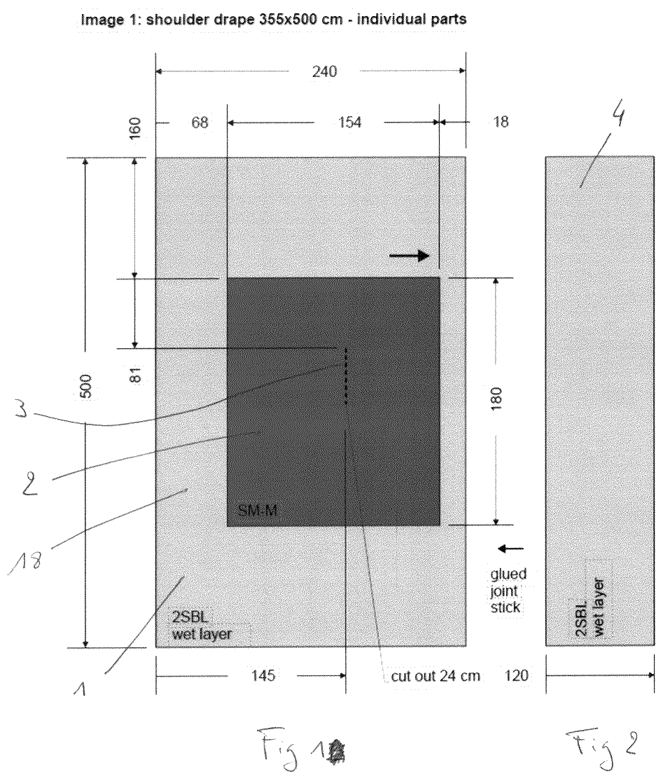

In a first aspect, the present invention relates to a surgical drape for sterile covering a patient for a shoulder surgery procedure. By way of illustration and not limited thereto, embodiments of the surgical drape are illustrated in FIGS. 1 to 5.

FIGS. 1 to 5 illustrate different parts which gradually create the surgical drape during manufacturing. FIG. 1 illustrates a relatively large first layer 1 from 2SBL material wherein 2SBL is a 2-ply laminated material comprising a lamination of hydrophilic spunbond and PE foil. This first layer has in the embodiment of FIG. 1 dimensions 240 cm×500 cm. In alternative embodiments these dimensions may be smaller or larger. They can be smaller or larger in one direction or in both directions. In alternative embodiments the first layer may be made of another material known in the art suitable for a sterile drape.

FIG. 1 further illustrates a second layer 2 which is glued on top of the first layer 1. In the embodiment of FIG. 1, the second layer 2 has smaller dimensions than the first layer 1. The second layer 2 has dimensions of 154 cm×180 cm. In alternative embodiments the dimensions of the second layer 2 may be smaller or larger, and may have the same or different dimensions than the first layer 1. The second layer 2 is made of SM-M material. SM-M material is an absorbent material. The second layer 2 is in the context of the current application also called the absorbing layer 2. In alternative embodiments the second layer may be made of another absorbent material known in the art suitable for a sterile drape.

After the second layer 2 is glued on the first layer 1, a cut 3 is made through both layers, thus through the second layer 2 and the first layer 1. In the embodiment of FIG. 1, the cut 3 is made over a straight line and has a length of 24 cm. In alternative embodiments the shape of the cut 3 may be different and/or the length of the cut 3 may be smaller or larger. The cut 3 is formed such that a sleeve 5 can be positioned in the cut 3. Therefore, the cut 3 is configured such that a sleeve 5 can be positioned and connected in or on the cut 3.

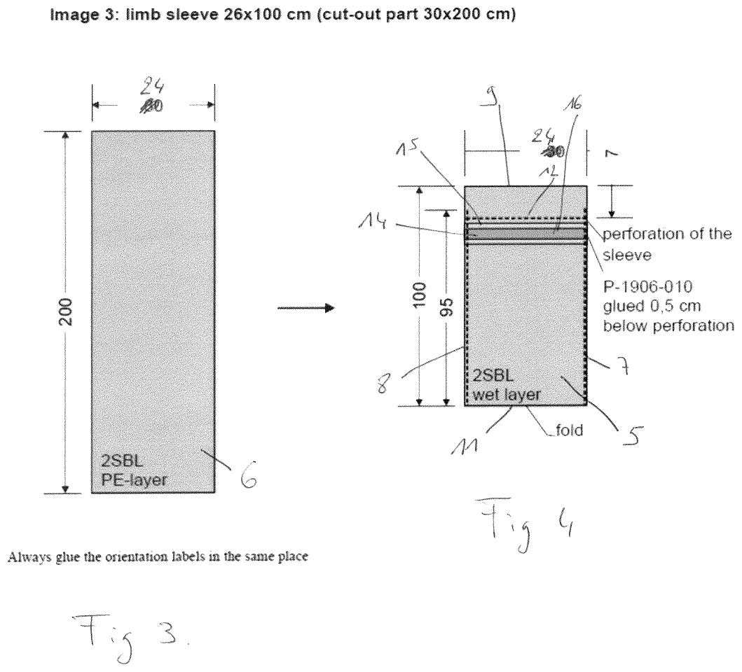

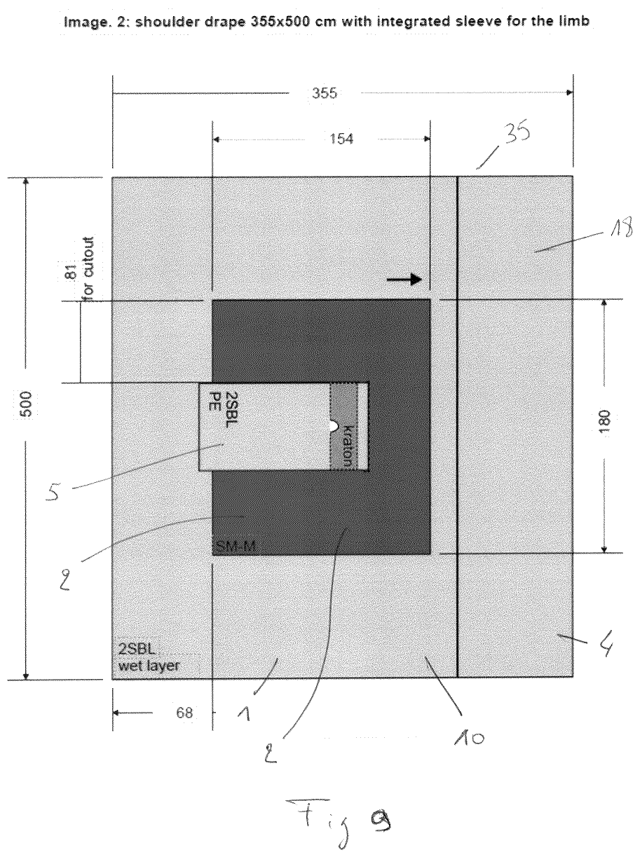

FIG. 2 shows a second part 4 made of the first layer material. The second part 4 is connected to the first layer 1 to create a larger first layer 10. The resulting larger first layer 10 is of the same material as the first layer. In the embodiment of FIGS. 1-2, this is of 2SBL material. In the embodiment of FIGS. 1-2, the width of the first layer 1 is made larger than 240 cm by gluing a second part 4 with a width of 120 cm to it forming a larger first layer 10 with a width of 355 cm. 5 cm of the combined width is used to glue forming an overlap of 2.5 cm. In alternative embodiments, the first layer 1 may not need an extension with a second part 4 to increase the width. In still an alternative embodiment the first layer is built up with more than two parts. The resulting first layer, independent if it is made out of a single unglued part or out of multiple parts glued together, is also called for the remaining of the description the main covering layer 10. The main covering layer 10 is shown in FIG. 9. The main covering layer 10 has a front surface 18 and a back surface 38. The back surface 38 is shown on FIG. 8.

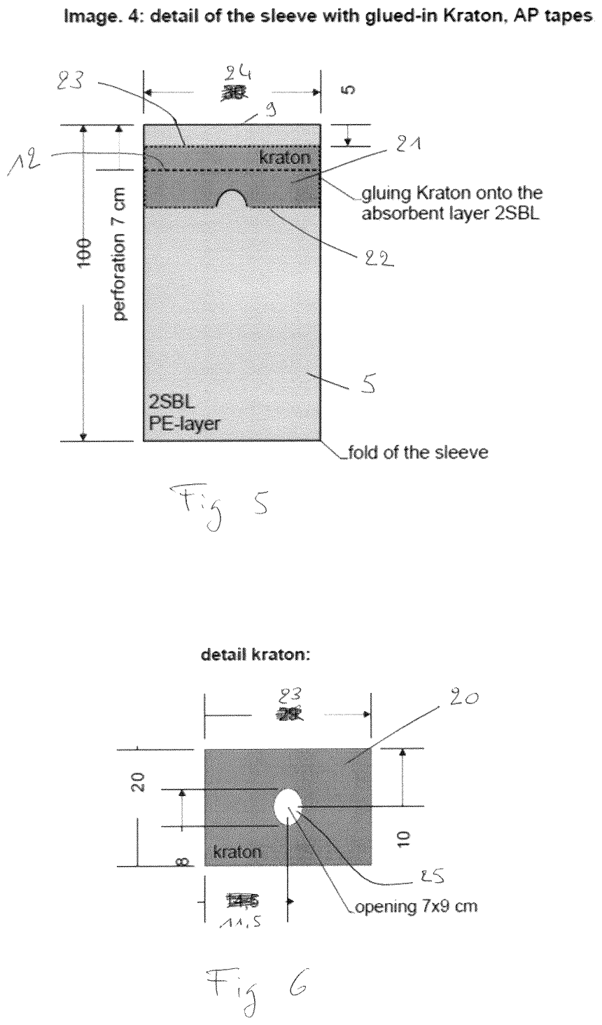

FIG. 3 illustrates a sleeve starting layer 6 for a limb sleeve 5 shown in FIG. 4. The sleeve starting layer 6 has in the embodiment of FIG. 3 a width of 24 cm and a length of 200 cm. The sleeve starting layer 6 is made of 2SBL material, the same material as the main covering layer 10. In alternative embodiments, the sleeve starting layer 6 may be larger or smaller. It can be larger or smaller in one or two directions or both. In alternative embodiments the sleeve starting layer 6 may be made of another material known in the art suitable for a sterile drape.

The sleeve starting layer 6 is subsequently folded in the length direction such that the folded sleeve starting layer 6 has still the same width, 24 cm in the embodiment of FIG. 4, and half of the length of before folding. In the embodiment of FIGS. 3-4 the folded length is 100 cm. The sides 7, 8 of the two folded parts are subsequently glued up to 5 cm of the open edge 9, i.e. the edge opposite to the folded edge 11. Therefore, in the embodiment where the folded length is 100 cm, the sides are glued over a length of 95 cm. In alternative embodiments, the sides of the folded sleeve starting layer 6 is glued up to a distance from the edge between 2 cm and 8 cm, preferably between 3 cm and 7 cm, more preferably between 4 cm and 6 cm, and most preferably at 5 cm. Further, the folded sleeve starting layer 6 is perforated over the width creating perforations 12 in both folded parts over the width. The perforations 12 are in the embodiment of FIG. 4 at 7 cm from the open edge 9. In alternative embodiments the perforations are at a distance between 5 cm and 9 cm from the open edge, preferably between 6 cm and 8 cm, and most preferred at 7 cm. In an alternative embodiment the perforations 12 are at a distance from the open edge 9 which is larger than or equal to the distance from the open edge that the sides are not glued. In alternative embodiments, the perforations are not forming a line but any other shape.

In alternative embodiments the distance where the gluing stops from the open edge 9 at the left side 8 is different from the distance where the gluing stops from the open edge 9 at the right side 7.

In some embodiments, the limb sleeve 5 may have a label 14 for providing an indication to a user where the perforations 12 are in the limb sleeve. In alternative embodiments, the label 14 may be any other means known in the art for providing an indication to a user. In the embodiment of FIG. 4, the label 14 is glued on the folded sleeve starting layer 6 over the width of the sleeve starting layer 6. The label 14 has two layers, a base label layer 15 and a central label layer 16. The base label layer 15 may have a height which is larger than the central label layer 16 as in the embodiment of FIG. 4. In alternative embodiments, the height of the base label layer 15 and the central label layer 16 may be the same. In still alternative embodiments, the height of the base label layer 15 may be smaller than the central label layer 16. In the embodiment of FIG. 4, the label 14 is glued on the sleeve starting layer 6 at a distance of 0.5 cm of the perforations. In alternative embodiments the label 14 may be positioned at another distance from the perforations, over the perforations or at the other side of the perforations. In alternative embodiments the label 14 has a different shape or may be printed on the sleeve starting layer. In some embodiments, the label 14 is at both sides of the folded sleeve starting layer. In other embodiments, the label 14 is at one side of the folded sleeve starting layer. In alternative embodiments the label 14 is at a distance from the perforations between 0 cm and 5 cm, preferably between 0 cm and 3 cm, more preferably between 0 cm and 1 cm, and most preferably at 0.5 cm.

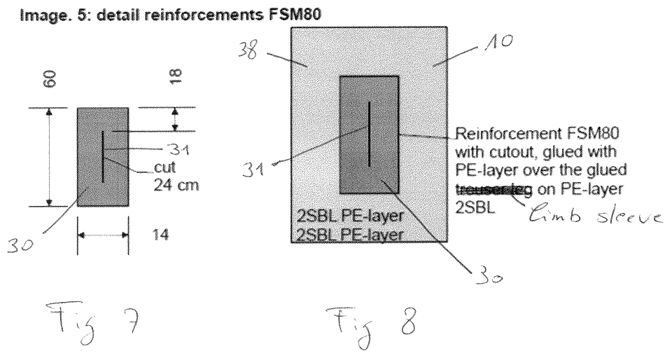

FIG. 6 illustrates an elastomer layer 20 which is prepared for gluing in the limb sleeve 5. In the embodiment of FIGS. 5-6 the elastomer layer 20 is made of Kraton. Kraton is a high-performance elastomer and a synthetic replacement for rubber. It has especially a high flexibility and traction. In alternative embodiments, the elastomer layer 20 may be from any other flexible material known in the art and useful for a sterile drape. In the embodiment of FIG. 6, the elastomer layer 20 has a width of 23 cm and a height of 20 cm. In the middle of the elastomer layer 20, an opening 25 of 7 cm×9 cm is provided. In alternative embodiments these dimensions may be smaller or larger. They can be smaller or larger in one direction or in both directions. The width of the elastomer layer 20 is in all embodiments suitable for assembling the elastomer layer in the limb sleeve 5.

The elastomer layer 20 is folded in the height direction forming a tourniquet element 21 which is assembled in the limb sleeve 5 as illustrated in FIG. 5. The tourniquet element 21 has a folded edge 22 through the opening 25. Opposite to the folded edge 22 is a connection edge 23. The tourniquet element 21 is assembled in the limb sleeve 5 at the connection edge 23. In the embodiment of FIG. 5, the tourniquet element 21 is glued to the sleeve starting layer 6 of the limb sleeve 5. In alternative embodiments, the tourniquet element 21 is assembled in the limb sleeve 5 by any other connection means known in the art.

The positioning of the tourniquet element 21 in the limb sleeve 5 is in the embodiment of FIG. 5 such that the connection edge 23 is at 5 cm from the open edge 9 of the limb sleeve and at 2 cm from the perforations 12. In alternative embodiments, the distance between the connection edge 23 and the open edge 9 is smaller or larger. In some embodiments, the connection edge 23 is positioned between the open edge 9 and the perforations 12. In alternative embodiments, the tourniquet element 21 is not connected in the sleeve but connected to the main covering layer 10.

The limb sleeve 5 is assembled to the main covering layer 10 on which the absorbing layer 2 is already glued as illustrated in FIG. 9. In alternative embodiments the absorbing layer 2 may be assembled after the limb sleeve 5 is assembled to the main covering layer 10. In the embodiment of FIG. 9, the limb sleeve 5 is positioned in the cut 3 and an end portion of the two folded parts of the limb sleeve is glued to the back surface of the main covering layer 10. The resulting surgical drape 35 is a drape comprising a limb sleeve 5, a tourniquet element 21, a main covering layer 10 and a absorbing layer 2 wherein part of the limb sleeve can be torn off from the rest of the surgical drape 35.

FIGS. 7 and 8 illustrates a reinforcement element 30 which is in some embodiments of the invention glued on the back surface of the main covering layer 10 after the limb sleeve 5 is assembled to the main covering layer 10. In the embodiment of FIG. 7, the reinforcement element 30 has a width of 14 cm and a length of 60 cm. In alternative embodiments these dimensions may be smaller or larger. They can be smaller or larger in one direction or in both directions. The reinforcement element 30 may be made of any material known in the art suitable for reinforcing a surgical drape. The reinforcement layer further has a cut 31 creating an opening which, in use, provides an opening for the patient to enter a limb through the opening in the limb sleeve.

As illustrated in FIG. 8, the reinforcement element 30 is glued on the main covering layer 10 over the glued limb sleeve portions which are already glued to the back surface of the main covering layer.

### Method of Preparing a Patient for a Shoulder Surgery Using a Surgical Drape According to an Embodiment of the Invention

In a second aspect, the present invention relates to a method of preparing a patient for shoulder surgery using a surgical drape 35 according to an embodiment.

The surgical drape 35 made according to one of the embodiments described above is folded to distribute and to use in an easy way. The surgical drape is folded with the opening on the back of the main covering layer accessible when folded and such that the limb sleeve 5, which is also folded, can unfold when a patient is entering a limb through the opening on the back of the main covering layer 10.

The folded surgical drape is positioned with the opening at the end of the limb that need to enter the limb sleeve for surgery. The surgical drape can subsequently be moved over the limb causing the limb sleeve to unfold. Once the limb is positioned in the limb sleeve, the main covering layer 10 is unfolded over the patient. The limb sleeve is now torn such that part of the limb sleeve from the perforations to the end is disconnected from the surgical drape. The disconnected limb sleeve part is moved over the limb such that a part of the limp becomes accessible. When the limp is sufficiently accessible, the disconnected limb sleeve part is hold in that position and a tape is applied on the disconnected limb sleeve part to keep it in the desired position. As a result, an accessible area is created with at the one side the tourniquet element 21 creating a circumferential pressure on the limb and on the other side the disconnected limb sleeve part kept in place by a tape. The advantage of this method is that a patient can be prepared for surgery in a minimum amount of steps. And only the surgical drape made as a single part and tape is needed for the preparation.

1. A surgical drape (35) for sterile covering of a patient for a shoulder surgery, comprising
a main covering layer (10) having a front surface (18) and a back surface (38),
an absorbing layer (2) applied to the front surface (18) wherein a first cut is made through the absorbing layer and the main covering layer, the cut being configured that the drape can move over an arm through the cut,
a limb sleeve (5) having a closed end and an open end wherein the limb sleeve is assembled to the main covering layer at the open end and the cut aligned such that when the drape is moved over the arm through the cut, the arm is covered by the limb sleeve, and
a tourniquet element (21) having an elastic opening configured to move over an arm and a connection edge assembled in the limb sleeve at a first distance from the open end,
wherein perforations are provided in the limb sleeve for tearing off part of the limb sleeve at a second distance from the open end, and
wherein first distance is lower than the second distance such that the tourniquet element is connected in the limb sleeve at a position between the open end of the limb sleeve and the perforations.

2. The surgical drape according to claim 1, wherein the surgical drape further comprises a reinforcement element (30), wherein the reinforcement element has a second cut, the second cut being configured that the drape can move over an arm through the second cut and wherein the reinforcement element is applied to the back surface of the main covering layer with the second cut aligned to the first cut such that the drape can move over an arm through the first cut and the second cut.

3. The surgical drape according to claim 1, wherein the perforations are at a distance between 5 cm and 9 cm from the open edge of the limb sleeve, preferably between 6 cm and 8 cm, and most preferred at 7 cm from the open edge of the limb sleeve.

4. The surgical drape according to claim 1, wherein the surgical drape further comprises a reinforcement element (30), wherein the reinforcement element has a second cut, the second cut being configured that the drape can move over an arm through the second cut and wherein the reinforcement element is applied to the back surface of the main covering layer with the second cut aligned to the first cut such that the drape can move over an arm through the first cut and the second cut, and wherein the perforations are at a distance between 5 cm and 9 cm from the open edge of the limb sleeve, preferably between 6 cm and 8 cm, and most preferred at 7 cm from the open edge of the limb sleeve.

5. The surgical drape according to claim 1, further comprising a means for indicating the location of the perforations, wherein the means for indicating the location of the perforations is provided on the limb sleeve.

6. The surgical drape according to claim 1, further comprising a means for indicating the location of the perforations, wherein the means for indicating the location of the perforations is glued on the limb sleeve.

7. The surgical drape according to claim 1 wherein the limb sleeve comprises a sleeve starting layer which is folded in the length direction and glued on both sides of the folded parts to form the sleeve with a closed end and an open end, wherein the folded parts are glued up to a first distance from the open end, and wherein the perforations are at a second distance from the open end wherein the second distance is larger than or equal to the first distance.

8. The surgical drape according to claim 1, wherein the limb sleeve is glued to the main covering layer.

9. The surgical drape according to claim 1, wherein the tourniquet element is glued in the limb sleeve.

10. A method of sterile covering of a patient for shoulder surgery using a surgical drape according to any one of the preceding claims 1 to 9 wherein the surgical drape is folded such that the limb sleeve can unfold without unfolding the main covering layer, comprising
moving the surgical drape through the first cut over an arm of a patient causing that the arm is covered by the limb sleeve,
unfolding the main covering layer over the patient,
tearing off part of the limb sleeve at the perforations,
moving the torn off part of the limb sleeve over the arm away from the shoulder to create an accessible area of desired size for the surgery,
fixing the position of torn off part of the limb sleeve on the arm when the desired size for the accessible area is reached.

11. The method of claim 10, wherein the fixing of the torn off part of the limb sleeve is executed by applying a tape on the torn off part of the limb sleeve.
